(19) United States
(12) Patent Application Publication
(10) Pub. No.: US 2010/0145355 A1
(43) Pub. Date: Jun. 10, 2010

(54) Circumcision Clamp

(76) Inventor: David R. Tomlinson, Wakefield, RI (US)

Correspondence Address: Peter J. Borghetti, 1002 Summer Ridge Court, Murrysville, PA, 15668, US

(21) Appl. No.: 12/592,136

(22) Filed: Nov. 19, 2009

Related U.S. Application Data
(63) Continuation-in-part of application No. 11/768,817, filed on Jun. 26, 2007.
(60) Provisional application No. 60/816,798, filed on Jun. 26, 2006.

(51) Int. Cl. A61B 17/326

(52) U.S. Cl. 606/118

(57) ABSTRACT

The present invention is a modification of a conventional circumcision clamp that prevents the use of mismatched parts.

### CROSS REFERENCE TO RELATED APPLICATIONS

The present invention is a Continuation In Part application of U.S. Non-Provisional application Ser. No. 11/768,817, entitled “CIRCUMCISION CLAMP” filed Jun. 26, 2007, which claims benefit of U.S. Provisional Application Ser. No. 60/816,798, entitled “SELF-ADJUSTING PRESSURE APPLICATOR” filed on Jun. 26, 2006, which both are incorporated herein by reference.

### FIELD OF THE INVENTION

One embodiment of the present invention is related generally to the field of circumcision.

### BACKGROUND OF THE INVENTION

Newborn circumcision is the most commonly performed surgical procedure in the World with an estimated 5 million circumcisions performed annually. Adult circumcisions are also performed routinely around the world. Circumcision has been performed for centuries for both religious and medical reasons. Circumcision instruments should, among other characteristics, be assembled with matching components to avoid possible penile amputation. For example, a conventional Gomco bell/stud can be lifted through the larger mismatched hole of the conventional Gomco plate and the incision can be made across the plate where the blade passes beneath the bell resulting in an amputation or partial amputation of the glans of the penis of the newborn child. In such cases of mismatched parts, the intended and necessary foreskin crushing action is not performed and hemorrhage can result.

### SUMMARY OF THE INVENTION

According to the invention, there is provided a circumcision clamp, as defined in claims 1-11.

For a better understanding of the present invention, together with other and further objects thereof, reference is made to the accompanying drawings and detailed description.

### BRIEF DESCRIPTION OF THE DRAWINGS

The present invention is illustratively shown and described in reference to the accompanying drawings, in which:

FIGS. 1A-D illustrates one embodiment of the present invention and its features that prevent the use of mismatched parts;

FIGS. 2A-B are photographs illustrating perspective views of a circumcision kit of the present invention with three (3) matched sets of plates, yolks, and ell/studs having the features that prevent the assembly of mismatched parts between matched plate, yolk, and bell/stud sets;

FIG. 2C is a photograph illustrating top views of the plates of the circumcision kit as shown in FIGS. 2A-B;

FIG. 2D is a photograph illustrating perspective views of the bell/studs of the circumcision kit as shown in FIGS. 2A-B;

FIG. 2E are top views of the front end of the plates illustrating the bell holes of the plates shown in FIG. 2C;

FIG. 2F are top views of the aft portion of the yokes of the circumcision kit as shown in FIGS. 2A-B;

FIGS. 2G-H are top views of matched yoke arms and bell/stud ends of the matched sets of the circumcision kit as shown in FIGS. 2A-B; and

FIGS. 2I-L are top views of portions of matched yoke bases and plates illustrating alternative positions, numbers and sizes of the holes and slots of the yoke base and plate.

### DETAILED DESCRIPTION OF THE INVENTION

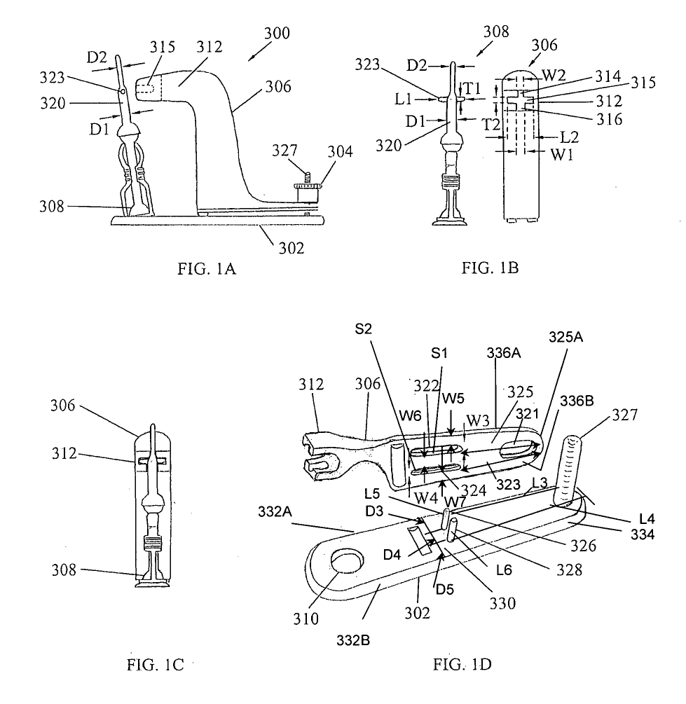

FIG. 1A-D shows one embodiment of the present invention clamp 300. Clamp 300 includes four parts: a plate 302, a nut 304, a yolk 306, and a bell/stud 308. Plate 302 includes a threaded member 327 that can cooperate with nut 304 with complimentary internal threads such that plate 302, yolk 306, and bell/stud 308 are secured in place once assembled as a unit. All four of these parts must be disassembled after each circumcision procedure for cleaning and sterilization and then reassembly to perform the next circumcision. The bell/stud 308 and plate 302 have many different sizes and are accordingly matched together. The present invention ensures that the unit can only be assembled with the appropriately sized parts. Any mismatched parts would prevent assembly and render the device unusable.

Additional embodiments of the matching devices of the present invention to ensure there is a correct fit between the bell/stud 308 and yolk 306 (FIG. 1B-C) includes two vertical slots 314 and 316 (yolk bell/stud matching device) formed in the front portion of the arm 312 of yolk 306. Slots 314 and 316 may have two different widths W1 and W2 that are sized to accept, for example, two vertical diameters D1 and D2 (bell/stud yolk matching device) near the distal end of the shaft 320 of the bell/stud 308. The shaft 320 on bell/stud 308 can only be positioned to work with arm 312 of yolk 306 if, for example, the shaft 320 diameters D1 and D2 were smaller then the widths W1 and W2 of slots 314 and 316, respectively, allowing bell/stud 308 to cooperate appropriately with yolk 306. For example, D1 is complimentary to W1 and D2 is complimentary to D2. It should be appreciated that though D1 is shown as being larger than D2, it is acceptable for D2 to be larger than D1. Also it is within the contemplation of the invention that one or more diameters of various diameter combinations can be formed in the shaft to act as a bell/stud yolk matching device depending, for example, on user needs.

FIG. 1C shows a front view of a matched bell/stud 308 received into the arm 312 of yolk 306, illustrating both the D1/D2 bell/stud yolk matching device and the cross member yolk matching device. However, the invention is not to be limited to the illustrated configuration that is being presented as an example and for convenience. It should be appreciated that that the unique bell/stud yolk matching devices and the yolk bell/stud matching devices can be used singular or in combination (as shown).

Other embodiments of the component matching devices of the present invention to ensure there is a correct fit between bell/stud 308 and yolk 306, shaft 320 can include a perpendicularly oriented cross member 323 (bell/stud yolk matching device) near the distal end of the shaft that includes a complimentary width (or thickness or diameter) and length such that cross member 323 is received into and enclosed within cross-ended horizontal slot 315 (yolk bell/stud matching device), as shown in FIG. 1C. For example, cross member 323 width T1 and length L1 are smaller than slot 315 width T2 and length L2. It should be appreciated that any dimensions of width and length are acceptable for cross member 323 and slot 315 as long as cross member 323 can be received by slot 315.

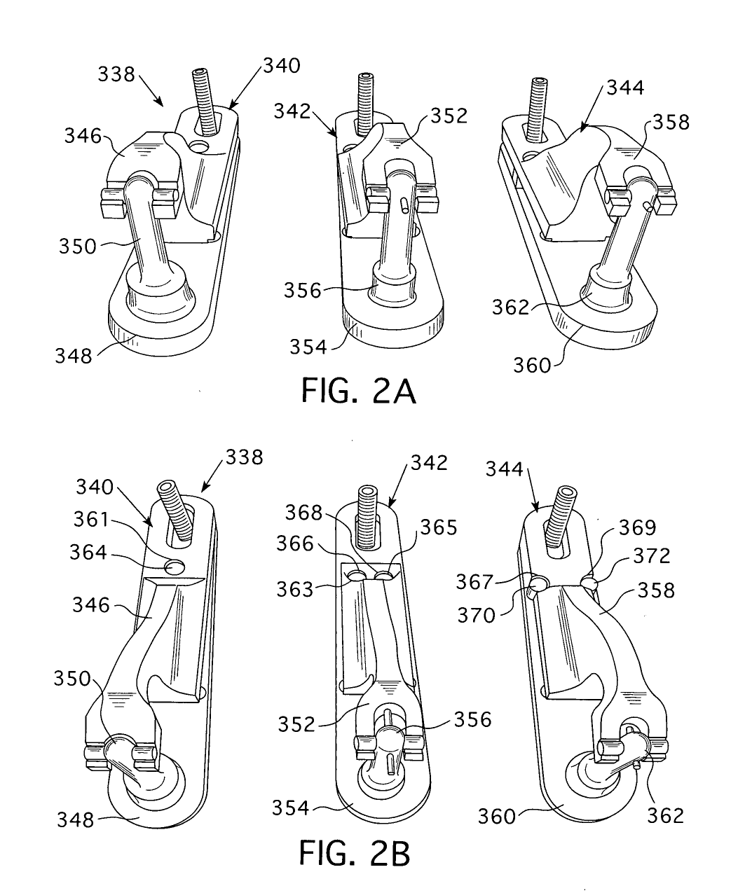

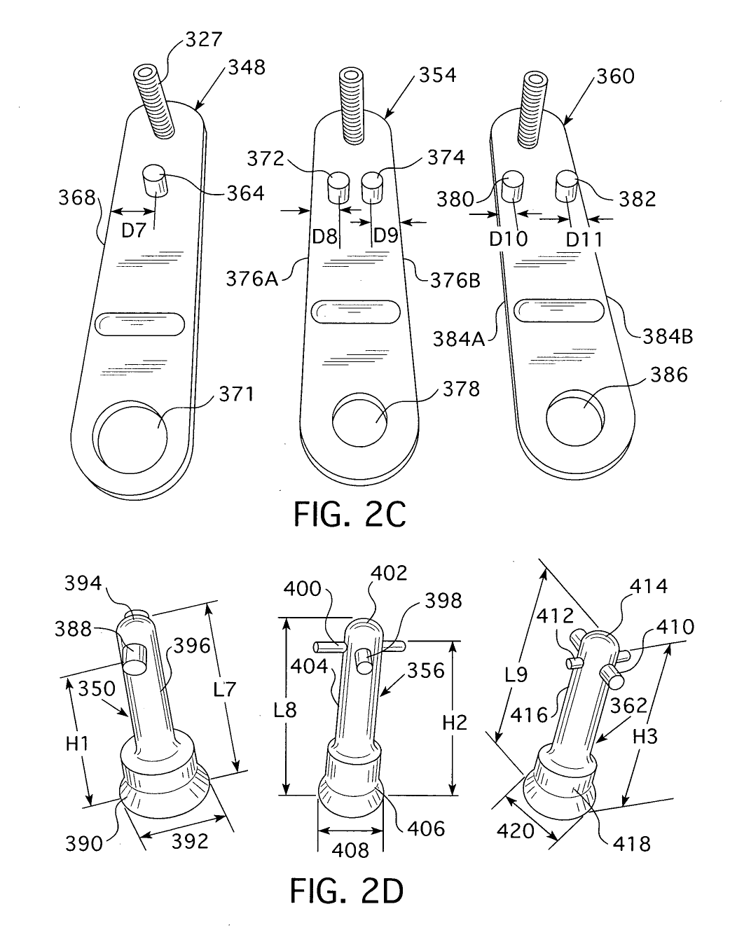

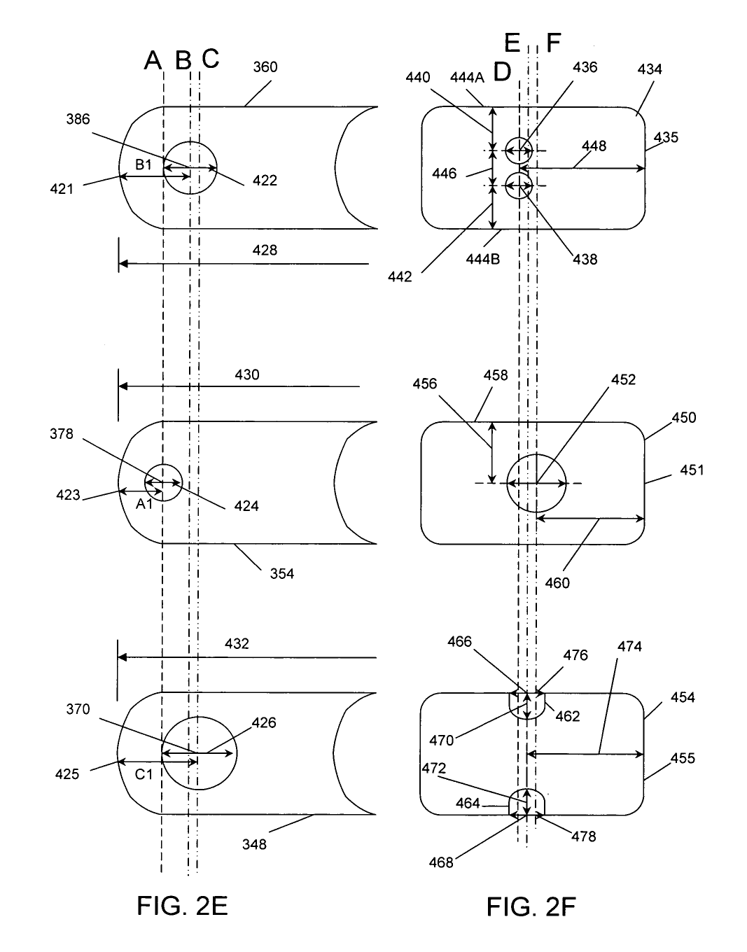

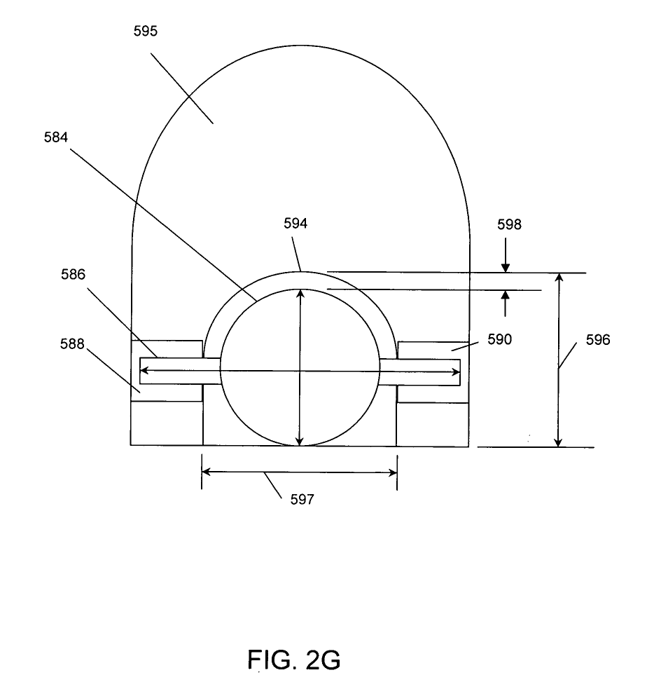

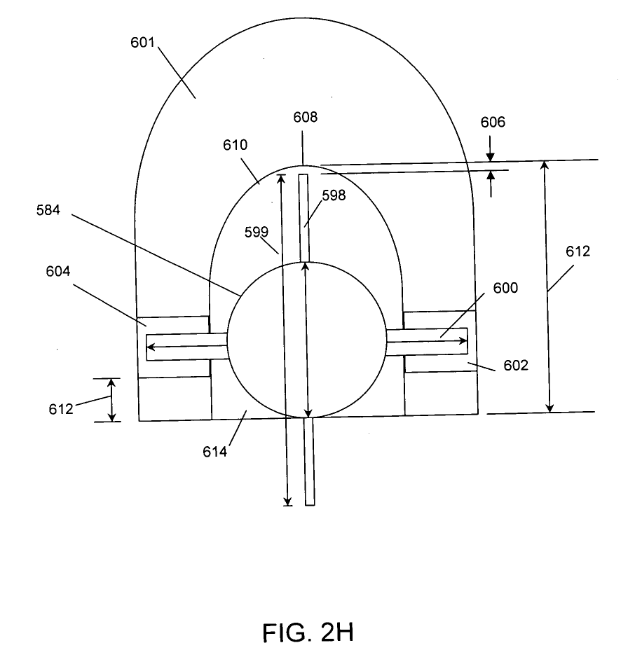

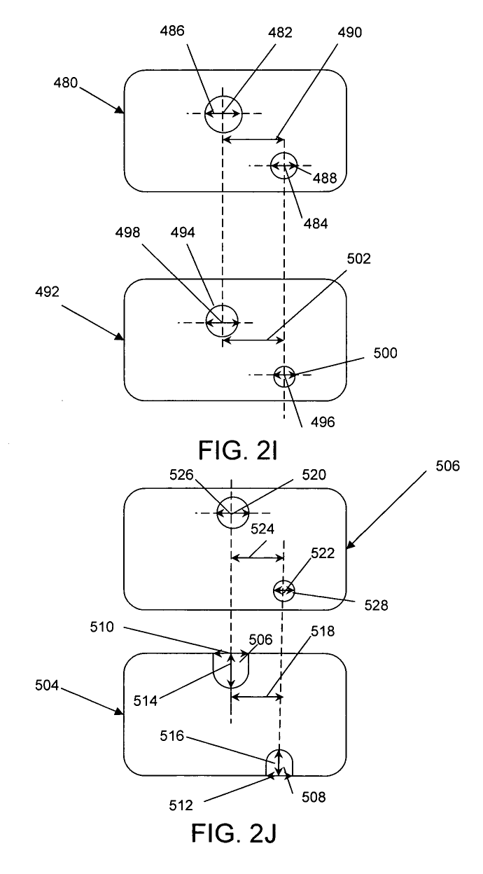

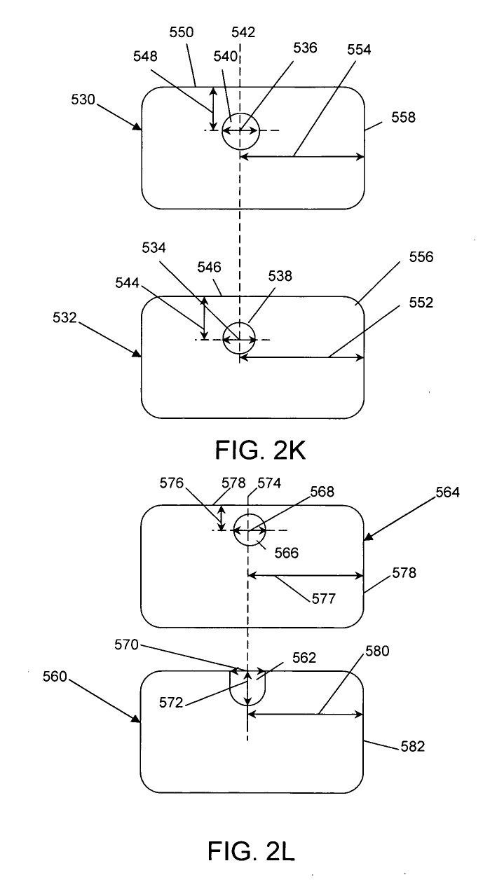

In yet another embodiment of the component matching device of the present invention to ensure there is a correct fit plate 330 and yolk 306. The embodiment shown in FIG. 1D includes two slots for illustration purposes but the invention is not to be limited to two or more apertures and posts. A single aperture and post as shown in FIGS. 2K-L is also contemplated within the scope of the invention. Now turning to FIG. 1D, yolk 306 can include two slots 322 and 324 on the bottom surface 325 of yolk 306 form a yolk plate matching device. Plate 302 can include two posts 326 and 328 that extend perpendicular and vertically from the top surface 330 of plate 302 to form a plate yolk matching device. Two posts 326 and 328 are spaced distances D3 and D5, respectively, from side edge 332A, 332B of plate 330 and distance D4 between posts 326 and 328. Two posts 326 and 328 are spaced Lengths L3 and L4, respectively, from back edge 334 of plate 330. Two posts 326 and 328 may have constant diameters over their lengths, L5 and L6, respectively, that are same or different that are uniquely sized to fit into the corresponding size slots 322 and 324 of the appropriately sized yolk 306. Slots 322 and 324 have widths W3 and W4 that are slightly larger then the corresponding diameters of posts 326 and 328. Slot 322 is a distance W5 from side edge 336A of plate 330. Slot 324 is a distance W4 from side edge 336B of plate 330. Slot 332 is a distance W6 from slot 324. Slots 322, 324 have predetermined lengths S1, S2 and positioned distances 321, 323 from back edge 325A of yolk 306. Lengths S1, S2 can be different. Distances 321 and 323 can be different. The position of the slots and posts are defined by widths W3, W4, W5, W6, distances D3, D4, D5, slot lengths S1, S2, and slot distances 321, 323. By varying the diameters and positions of the posts 326 and 328 on plate 302 and varying the width and position of the slots 322 and 324 between different sized parts, same size parts can be uniquely fitted so that only the correctly sized parts can successfully mate allowing the device to work and that any other combination can not mate, prohibiting the use of the device. The vertical design of the posts 326 and 328 allows a mated yolk 306 to move as is necessary to perform its clamping function. When posts 326 and 328 of a plate 302 mate with the corresponding slots 322 and 324 of yolk 306, any lateral movement of the yolk 306 in relation to the plate 302 is prevented. This ensures the arm 312 maintains its correct position centered over the thru hole 310 of plate 302, providing a further benefit to the user. It should be appreciated that any diameter of post 326, 328 and any widths of slots 322, 324 are acceptable for the plate yolk matching device and the yolk plate matching device, respectively.

Another embodiment of the invention is a circumcision kit 338 illustrated in FIGS. 2 A-B. Circumcision kit 338 is shown with three (3) matched sets of yokes, plates, and bells. However, the invention is not to be limited by the number of matched sets. First matched set 340 includes uniquely configured yoke 346, plate 348, and bell/stud 350. Second matched set 342 includes uniquely configured yoke 352, plate 354, and bell/stud 356. Third matched set 344 includes uniquely configured yoke 382, plate 360, and bell/stud 362. As shown in FIG. 2B, each match set includes a plate/yoke matching device of a yoke aperture 361, 363, 365, 367, 369 sized to receive a plate post, 364, 366, 368, 370, 372.

FIG. 2C illustrates three (3) plates of circumcision kit 338. Plate 348 includes one post 364 having a distance D7 from side edge 368 and a bell hole 371. Plate 354 includes two posts 372, 374 having distances D8, D9 from side edge 376A, 376B and a bell hole 378. Plate 360 includes two posts 380, 382 having distances D10, D11 from side edge 384A, 384B and a bell hole 386. As described above, diameters and positions of posts align with apertures of a matching yoke.

FIG. 2D illustrates three (3) bell/studs of circumcision kit 338. Bell/stud 350 having overall length L7, one cross-member 388 in shaft end 392 of shaft 396 being height H1 from bell base 390, and bell diameter 392 being larger than bell hole 371 of matching plate 348. Bell/stud 356 having overall length L8, cross-member 398 and restraint pin 400 in shaft end 402 of shaft 404 being height H2 from bell base 406, and bell diameter 408 being larger than bell hole 378 of matching plate 354. Though, cross-member 398 and restraint pin 400 are shown at same height, they can be at different heights. Bell/stud 362 having overall length L9, cross-member 410 and restraint pin 412 in shaft end 414 of shaft 416 being height H3 from bell base 418, and bell diameter 420 being larger than bell hole 386 of matching plate 360. Though, cross-member 410 and restraint pin 412 are shown at same height, they can be at different heights.

FIG. 2E illustrates various configurations of the plate bell hole diameters and positions. Bell hole diameters 422, 424, 426 can be different. Centerlines A, B, C of bell hole diameters 422, 424, 426 can be positioned from edge 421, 423, 425 at different distances B1, A1, C1. Lengths 428, 430, 432 of plates 360, 354, 348 can be different.

FIG. 2F illustrates three (3) yolk plate matching device aperture sizes and positions that match with the corresponding plates of circumcision kit 338. For illustrations purposes, yoke 450 and plate 348 is first matched set, yoke 434 and plate 354 is second matched set 342, and yoke 454 and plate 360 is third matched set 344. Yoke 434 includes two apertures 436, 438 having distances 440, 442 from side edge 444A, 444B, distance 446 between two apertures 436, 438, and distance 448 from aperture centerline D to yoke edge 435. Two apertures 436, 438 are shown with the same diameter but can be different diameters and/or be on different centerlines, for example centerlines are offset (see FIG. 2I). Yoke 450 includes one aperture 452 having distance 456 from side edge 458, and distance 460 from aperture centerline F to yoke edge 451. Yoke 454 includes two apertures 462, 464 with slot lengths 466, 468 and depths 470, 472 and distance 474 from aperture centerline E to yoke edge 455. Two apertures 462, 464 have openings 476, 478. Two apertures 462, 464 are shown with the same slot lengths and depths but can be different dimensions and/or be on different centerlines, for example centerlines are offset (see FIG. 2J).

Now turning to FIG. 2I illustrating one embodiment of a yolk base 480 and plate 492 matched set of the present invention with offsetting centering lines of apertures and different size diameters and slots. Yolk base 480 includes two apertures 482, 484 having different diameters 486, 488 and being longitudinally offset 490. Plate 492 includes two posts 494, 496 having different diameters 498, 500 and being longitudinally offset 502. Diameters 486, 488 of two apertures 482, 484 of yolk base 480 are larger than diameters 498, 500 of two posts 494, 496 of plate 492. The remaining features of yolk base 480 and plate 492 are the same as the features of yolk 434 and plate 354 described and shown in FIG. 2F and FIG. 2C, respectively. Offsets 490, 502 are substantially the same.

Now turning to FIG. 2J illustrating another embodiment of a yolk base 504 and plate 506 matched set of the present invention with offsetting centering lines of apertures and different size diameters and slots. Yolk base 504 includes two apertures 506, 508 having slot lengths 510, 512 and depths 514, 516 being longitudinally offset 518. Slot lengths 510, 512 and/or depths 514, 516 can be the same (not shown) or different (as shown in FIG. 2J). Plate 506 includes two posts 520, 522 diameters 526, 528 and being longitudinally offset 524. Diameters 526, 528 can be the same (not shown) or different (as shown in FIG. 2J). Slot lengths 510, 512 of two apertures 506, 508 of yolk base 504 are respectively larger than diameters 526, 528 of two posts 520, 522 of plate 506. The remaining features of yolk base 504 and plate 506 are the same as the features of yolk 454 and plate 360 described and shown in FIG. 2F and FIG. 2C, respectively. Offsets 518, 524 are substantially the same.

Now turning to FIG. 2K illustrating another embodiment of a matched yolk and plate of the present invention with a yolk 530 having a single aperture 536 and a plate 532 with a single post 534. Diameter 538 of single post 534 is smaller than diameter 540 of single aperture 536. Centerline 542 is common with single post 534 of plate 534 and single aperture 536 of yoke 530. Center of single post 534 of plate 532 is a distance 544 from side edge 546 of plate 532. Center of aperture 536 of yolk 530 is a distance 548 from side edge 550 of yolk 530. Center of single post 534 of plate 532 is a distance 552 from back edge 556 of plate 532. Center of aperture 536 of yolk 530 is a distance 554 from back edge 558 of yolk 530. By relatively changing the distances 544, 548, 552, 554, different matching patterns can be developed such that no two matched sets of yolk and plate have the same aperture/post configuration.

Now turning to FIG. 2L illustrating another embodiment of a matched yolk and plate of the present invention with a yolk 560 having a single aperture 562 and a plate 564 with a single post 566. Diameter 568 of single post 566 is smaller than width 570 and depth 572 of single aperture 562. Centerline 574 is common with single post 566 of plate 564 and single aperture 562 of yoke 560. Centerline of single post 566 of plate 564 is a distance 576 from side edge 578 of plate 564. Centerline of single post 566 of plate 564 is a distance 577 from back edge 578 of plate 564. Centerline of aperture 562 of yolk 560 is a distance 580 from back edge 582 of yolk 560. By relatively changing the distances 576, 577 and slot depth 572, distance 580, different matching patterns can be developed such that no two matched sets of yolk and plate have the same aperture/post configuration. Also, varying aperture diameters and slot lengths and depths will create different matching patterns.

Now turning to FIGS. 2G-H illustrating top views of embodiments of bell shaft/yolk matching device. FIG. 2G illustrates a bell/stud shaft end 584 with a cross member 586 having length 589 that rests in grooves 588, 590 and diameter 592 that is received into recess 594 of yolk arm 595 with depth 596 creating gap 598 between bell/stud shaft end 584 and recess 594. Recess entrance width 597 and shaft diameter 592 can be varied to create different matching patterns, wherein shaft diameters greater than recess entrance width will not fit within the recesses.

FIG. 2H further includes an inference pin 598 having length 599 that will not allow cross member 600 to rest within grooves 602, 604 of yolk arm 601 without gap 606 being formed between bottom 608 of recess 610 and inference pin 598. Grooves 602, 604 are positioned a distance 612 from recess entrance 614. Varying any and all combinations of depth 612 of recess 610, length 599 of inference pin 598, and distance 612 from recess entrance 614 can create different matching patterns. Inference pin 598 and cross member 600 can either by in the same lateral plane or not.

Further embodiments of bell/stud 308 are disclosed in U.S. patent application Ser. No. 11/768,808 filed on Jun. 26, 2007, “Self-Adjusting Pressure Applicator,” by David R. Tomlinson, and U.S. patent application Ser. No. 11/571,120 filed on Dec. 21, 2006, “Atraumatic Circumcision Device and Method to Use,” by David R. Tomlinson, which is a U.S. national phase entry of Patent Cooperation Treaty international application serial number PCT/US2005/022404 filed on Jun. 23, 2005, “Atraumatic Circumcision Device and Method to Use,” by David R. Tomlinson, which claims priority to U.S. provisional application Ser. No. 60/583,259 filed on Jun. 25, 2004, “Atraumatic Circumcision Ring and Method,” by David R. Tomlinson, whereby all above listed applications are herein incorporated by reference.

While the disclosure has been described in detail and with reference to specific embodiments thereof, it will be apparent to one skilled in the art that various changes and modifications can be made therein without departing from the spirit and scope of the embodiments. Thus, it is intended that the present disclosure cover the modifications and variations of this disclosure provided they come within the scope of the appended claims and their equivalents.

What is claimed is:

1. A circumcision kit comprising:
a first matched plate and yolk set includes a first plate, a first yolk, and a first bell/stud;
wherein the first plate having at least one post extending substantially perpendicular and substantially vertical from a top surface of the first plate at a predetermined position, wherein the at least one post of the first plate has a constant diameter over a length of the least one post of the first plate;
wherein the first yolk includes at least one aperture having (i) a width larger than the constant diameter of the at least one post of the first plate, and (ii) a predetermined position aligned with the predetermined position of the at least one post of the first plate; and
a second matched plate and yolk set includes a second plate, a second yolk, and a second bell/stud;
wherein the second plate having at least one post extending substantially perpendicular and substantially vertical from a top surface of the second plate at a predetermined position, wherein the at least one post of the second plate has a constant diameter over a length of the least one post of the second plate;
wherein the second yolk includes at least one slot having (i) a width larger than the constant diameter of the at least one post of the second plate, and (ii) a predetermined position aligned with the predetermined position of the at least one post of the second plate; and
wherein either:
(i) the predetermined position of the least one post of the first plate is not the same as the predetermined position of the least one post of the second plate, or
(ii) the diameter of the least one post of the first plate is larger than the width of the at least one slot of the second yolk,
whereby the first plate cannot be fitted properly with the second yolk and the second plate cannot be fitted properly with the first yolk.

2. The circumcision kit according to claim 2 further comprising:
a third matched plate and yolk set includes a third plate, a third yolk, and a third bell/stud;
wherein the third plate having at least one post extending substantially perpendicular and substantially vertical from a top surface of the third plate at a predetermined position, wherein the at least one post of the third plate has a constant diameter over a length of the least one post of the third plate;
wherein the third yolk includes at least one slot having (i) a width larger than the constant diameter of the at least one post of the third plate, and (ii) a predetermined position aligned with the predetermined position of the at least one post of the third plate; and
wherein either:
(i) the predetermined position of the least one post of the third plate is not the same as at least one of the following: the predetermined position of the least one post of the first plate or the predetermined position of the least one post of the second plate, or
(ii) the diameter of the least one post of the third plate is larger than at least one of the following: the width of the at least one slot of the first yolk or the width of the at least one slot of the second yolk,
whereby the third plate cannot be fitted properly with the first yolk and the second yolk, and the third yolk cannot be fitted properly with the first plate and the second plate.

3. The circumcision kit according to claim 2 wherein:
wherein the first plate further comprises a second post extending substantially perpendicular and substantially vertical from the top surface of the first plate at a predetermined position different than the predetermined position of the at least one post of the first plate, wherein the second post of the first plate has a constant diameter over a length of the second post of the first plate;
wherein the first yolk further comprises a second slot having (i) a width larger than the constant diameter of the second post of the first plate, and (ii) a predetermined position aligned with the predetermined position of the second post of the first plate.

4. The circumcision kit according to claim 3 wherein:
wherein the second plate further comprises a second post extending substantially perpendicular and substantially vertical from the top surface of the second plate at a predetermined position different than the predetermined position of the at least one post of the second plate, wherein the second post of the second plate has a constant diameter over a length of the second post of the second plate;
wherein the second yolk further comprises a second slot having (i) a width larger than the constant diameter of the second post of the second plate, and (ii) a predetermined position aligned with the predetermined position of the second post of the second plate; and
wherein either:
(i) the predetermined position of the second post of the second plate is not the same as the predetermined position of the second post of the first plate, or
(ii) the diameter of the second post of the second plate is larger than the width of the second slot of the first yolk,
whereby the first plate cannot be fitted properly with the second yolk and the second plate cannot be fitted properly with the first yolk.

5. The circumcision kit according to claim 2 wherein:
wherein the third plate further comprises a second extending substantially perpendicular and substantially vertical from the top surface of the third plate at a predetermined position different than the predetermined position of the at least one post of the third plate, wherein the second post of the third plate has a constant diameter over a length of the second post of the third plate;
wherein the third yolk further comprises a second slot having (i) a width larger than the constant diameter of the second post of the third plate, and (ii) a predetermined position aligned with the predetermined position of the second post of the third plate; and
wherein either:
(i) the predetermined position of the second post of the third plate is not the same as one of the following: the predetermined position of the least one post of the first plate, the predetermined position of the least one post of the second plate, the predetermined position of the second post of the first plate, or the predetermined position of the second post of the second plate, or
(ii) the diameter of the third post of the third plate is larger than one of the following: the width of the slot of the first yolk, the width of the second slot of the first yolk, the width of the slot of the second yolk, or the width of the second slot of the second yolk,
whereby the third plate cannot be fitted properly with the first yolk and the second yolk, and the third plate cannot be fitted properly with the first yolk and the second yolk.

6. A circumcision kit comprising:
a first matched set includes a first yolk, a first bell/stud, and a first plate wherein, (i) the first plate includes a single post having a diameter at a first predetermined distance from an edge of the first plate and a threaded member, and (ii) the first yolk includes an aperture having a diameter at a first predetermined distance from an edge of the first yolk, wherein the single post diameter is smaller than the first yolk aperture diameter;
a second matched set includes a second yolk, a second bell/stud, and a second plate wherein, (i) the second plate includes a first post having a first diameter at a second predetermined distance from an edge of the second plate, a second post having a second diameter at a third predetermined distance from the edge of the second plate, and a threaded member, (ii) and the second yolk includes a first aperture having a diameter at a second predetermined distance from an edge of the second yolk, a second aperture having a second diameter at a third predetermined distance from the edge of the second yolk, wherein the first post diameter is smaller than the first aperture diameter and the second post diameter is smaller than the second aperture diameter; and
a third matched set includes a third yolk, a third bell/stud, and a third plate wherein, (i) the third plate includes a first post having a first diameter at a fourth predetermined distance from an edge of the third plate, a second post having a second diameter at a fifth predetermined distance from the edge of the second plate, and a threaded member, (ii) and the second yolk includes a first aperture having a diameter at a fourth predetermined distance from an edge of the second yolk, a second aperture having a second diameter at a fifth predetermined distance from the edge of the second yolk, wherein the first post diameter is smaller than the first aperture diameter and the second post diameter is smaller than the second aperture diameter.

7. The circumcision kit according to claim 6 wherein the second bell/stud includes a cross member and an inference pin.

8. The circumcision kit according to claim 6 wherein the third bell/stud includes a cross member and an inference pin.

9. The circumcision kit according to claim 6 wherein the first, second, third, fourth, and fifth predetermined locations of the pins are different.

10. The circumcision kit according to claim 6 wherein the second bell/stud inference pin includes a length difference than a length of the third bell/stud inference pin.

11. The circumcision kit according to claim 6 wherein includes the first and the second apertures of the second yolk each have an opening on an edge of the yolk.
